(19) United States
(12) Patent Application Publication
(10) Pub. No.: US 2016/0235974 A1
(43) Pub. Date: Aug. 18, 2016

(54) CANCEROUS METABOLIC GROWTH INHIBITOR SYSTEM

(71) Applicant: AngioDynamics, Inc., Latham, NY (US)

(72) Inventors: Kevin Holochwost, Hudson, MA (US); Paul DiCarlo, Middleboro, MA (US)

(73) Assignee: AngioDynamics, Inc., Latham, NY (US)

(21) Appl. No.: 14/620,773

(22) Filed: Feb. 12, 2015

(51) Int. Cl. A61N 1/20

(52) U.S. Cl. CPC A61N 1/205

(57) ABSTRACT

A device and method to inhibit growth of cancer cells is disclosed. The device may include a power source and an electric field generator. The power source may output an electric potential to cause an electric field to be emitted from the electric field emitter and be applied to target tissue, which may include cancer cells.

### FIELD OF THE DISCLOSURE

The disclosure relates generally to the field of cancer treatment, and more particularly to cancer growth inhibitors.

### BACKGROUND OF THE DISCLOSURE

Generally speaking, cancer is an abnormal growth of cells, which can affect both human and animal bodies. Abnormal cell growth can take many forms and affect various tissues of a body. The goal for cancer treatment is the removal of the cancerous cells (e.g., tumorous growth, etc.) without extensive damage to the rest of the body. Modem cancer treatments include a range of options, such as, for example, radiation, hormones, surgery, as well as other treatment modalities.

Some cancers are known to grow slowly, for example prostate cancer, which may be untreated for many years while being monitored. Other cancers may spread rapidly from organ to organ necessitating aggressive treatments that must be delivered in a timely fashion. Furthermore, some cancers have low, survival rates and no elective treatment exists beyond intervention to extend or improve quality of life.

Slowing cancerous cell growth can be beneficial to a large number of cancer treatment options. For example, slowing the growth of cancerous tissue may allow for the extension of the patient's life and may provide an increase in the quality of life for the patient. Additionally, slowing the growth of cancerous tissue may provide an increase in the time allowed to prepare for or perform other treatment procedures, which may increase survival rates.

One reason that cancer cells grow abnormally with respect to normal cells is that cancer cells do not metabolize using the same mechanisms as a normal cell. The primary cell organelle associated with such processes is the mitochondria. FIG. 1 illustrates an example of a mitochondrion 100. As can be seen, the mitochondrion 100 has an outer membrane 110. Within the outer membrane 110 is an inner membrane 120 that encloses the chemiosmotic apparatus (e.g., deoxyribonucleic acid, ribosome, adenosine triphosphate synthase, etc.). The outer membrane 110 and inner membrane 120 are separated by an intermembrane space 130.

The outer membrane 110 includes pores that allow molecules of approximately several thousand (atomic mass units) AMUs to move freely between the inner and outer portions of the organelle membrane. The inner membrane 120, unlike the outer membrane 110, does not contain “pores.” Accordingly, most molecules require transport to move across the inner membrane 120. A voltage potential across the inner membrane 120 allows for electron train transport to move molecules from within the intermembrane space 130 into and through the inner membrane 120. Interruption of this electron train transport may disrupt the function of the mitochondria 100 and may lead to cell death (e.g., by allowing proteins to leak from the intermembrane space 130 into the cytosol (not shown), or the like). It is important to note, that interruption of electron train transport in any cell (e.g., normal, healthy, cancerous, or the like) can lead to cell death.

The electrical properties of cells, and particularly, cancer cells are described in greater detail in Electrical Properties of Cancer cells, Published online at http://www.royalrife.com/haltiwanger1.pdf, by Steve Haltiwanger M.D., C.C.N; and Membrane Potential and Cancer Progression, Frontiers in Physiology 4, July 2013, which articles are incorporated entirely herein by reference.

Some pharmaceutical based treatments that target mitochondrial metabolism have been proposed, such as, for example, in Mitochondrial Metabolism Inhibitors for Cancer Therapy, Journal of Pharmaceutical Research, November 2011, Volume 28, Issue 11, pp. 2731-2744, which article is incorporated entirely herein by reference. However, pharmaceutical treatments are often delivered systemically. As such healthy, non-target tissues, are exposed to the drug, which may result in significant side effects including nausea, vomiting, hair loss, fatigue and increased probability of infection due to low white blood cell counts. Accordingly, there is a need for non-drug based treatments that can be used by themselves or in conjunction with other drug based treatments.

Non-drug based treatments that target electrical functioning of cells have been proposed in U.S. Pat. No. 8,656,930, entitled “Method and System for Processing Cancer Cell Electrical Signals for Medical Therapy” to E. Schuler et al.; and U.S. Pat. No. 8,831,738, entitled “System and Method to Elicit Apoptosis in Malignant Tumor Cells for Medical Treatment” to E. Schuler et al., which patents are incorporated entirely herein by reference. Such conventional non-drug based treatments are complex and require a significant amount of a priori knowledge about the particular type of cancer cell targeted for treatment. In particular, these conventional systems require significant monitoring of cancerous cells and substantial individual design to develop electrical signals that are intended to “reprogram” cell functioning of cancerous cells. As such, these treatments are not readily adaptable to slowing cancerous cell growth.

It is with respect to these and other considerations that the present improvements are needed.

### SUMMARY

In view of the forgoing, device to inhibit growth of cancerous cells is disclosed. The device may implantable into a living subject or may be wearable by a living subject. The device may include an electric field generator operably coupled to the power source, and a housing that contains the power source and at least a portion of the electric field generator; wherein the power source generates an electric potential of 2.0 Volts to 10.0 Volts; and wherein the electric field generator generates an electric field that extends outside of the housing and includes at least a portion of the cancer cell.

### BRIEF DESCRIPTION OF THE DRAWINGS

By way of example, specific embodiments of the disclosed device will now be described, with reference to the accompanying drawings, in which;

FIG. 1 illustrates a mitochondrion.

FIG. 2 illustrates an embodiment of a cancer cell growth inhibitor device.

FIG. 3 illustrates an electric field applied to a cell wall.

FIG. 4 illustrates an electric field applied to a healthy cell wall.

FIG. 5 illustrates an electric field applied to a cancer cell wall.

FIGS. 6A-6B illustrate the depth of penetration of an electric field through target tissue.

FIGS. 7A-7B illustrate another embodiment of a cancer cell growth inhibitor device.

FIG. 8 illustrates an example of a cancer cell growth inhibitor device implanted in a patient.

FIG. 9 illustrates another embodiment of a cancer cell growth inhibitor device.

FIG. 10 illustrates an example of multiple cancer cell growth inhibitor devices implanted in a patient.

FIGS. 11A-11B illustrate another embodiment of a cancer cell growth inhibitor.

FIG. 12 illustrates a method of treating cancer cells.

### DETAILED DESCRIPTION

Various examples, implementations, and illustrative configurations are described herein. It is to be appreciated that the various depictions are not drawn to scale; instead, they are drawn in a manner to facilitate understanding. Additionally, the various examples and illustrations can be combined with each other, even where not specifically so stated. Additionally, the described examples are not intended to limit the claims; instead, the present disclosure is presented such that the claimed subject matter will be thoroughly illuminated.

The present disclosure provides an apparatus and/or method to inhibit the growth of cancer cells. In particular, the present disclosure provides a device configured to apply an electrical field to tissue that may include target cells (e.g., cancerous cells, or the like). Application of an electric field to tissue that includes target cells, and how this electrical field may slow the growth of the target cells is described in greater detail below. However, in general, the device is configured to apply an electric field to the tissue, where the electric field is configured such that it penetrates the cell walls of the target cells to disrupt or inhibit the function of the target cells, thereby causing reduced reproduction and/or cell death.

It is to be appreciated, that the present disclosure may be applied to tissue that includes both target cells (e,g, cancerous cells) and non-target (e.g., healthy cells, non-cancerous cells, or the like). The device may apply an electric field to the tissue to inhibit the growth of the target cells at a greater rate and/or within a larger area that it affects the growth of the non-target cells. The affect of the electric field on the target cells and the non-target cells is described in greater detail below. However, application of the electric field may disrupt the function of the target cells in a much larger region of the tissue than it disrupts the function of the non-target cells. As such, the present disclosure may be applied to inhibit the growth of the target cells (e.g., the cancerous cells) without substantially affecting the functioning of the non-target cells (e.g., healthy cells).

Additionally, it is important to note, the present disclosure may be implemented to apply an electric field to transport electrical current through existing ion channels of a cell to inhibit growth and reproduction of the cell. Conversely, some conventional technologies, such as, for example, electroporation, open cell pores (either permanently or temporarily) to disrupt the adenosine triphosphate (ATP) cycle of the cell.

As a result, application of an electrical field to cancerous tissue according to the present disclosure may slow the growth (e.g., multiplication, regeneration, or the like) of new target cells within the tissue, thereby allowing for the determination of optimum treatments, extending life, improving quality of life and care, lowering healthcare related costs for patient's and hospitals, simplifying treatment regimes, and/or improving clinical outcomes from other treatment modalities. In some examples, the present disclosure may be utilized as a primary cancer treatment modality. With some examples, the present disclosure may be utilized for post-surgical or post-primary modality treatment. For example, the present disclosure may be utilized for the prevention and slowing of secondary cancerous growth, tumor seeding, or same site recurrence.

In some examples, the treatment options disclosed herein may be used along with (e.g., as a primary treatment, as a secondary treatment, or the like) other treatment modalities e.g., surgery, radiation, chemotherapy, or the like). For example, the present disclosure may be applied along with a lumpectomy to treat breast cancer. As another example, the present disclosure may be applied as a primary treatment to slow the growth of prostate cancer while the cancer is monitored.

In some examples, the device may be implemented such that it can be implanted into a patient's body to apply an electrical field from an internal position within the patient's body. With some examples, the device may be implemented such that it can be worn to apply an electrical field from an external position outside the patient's body.

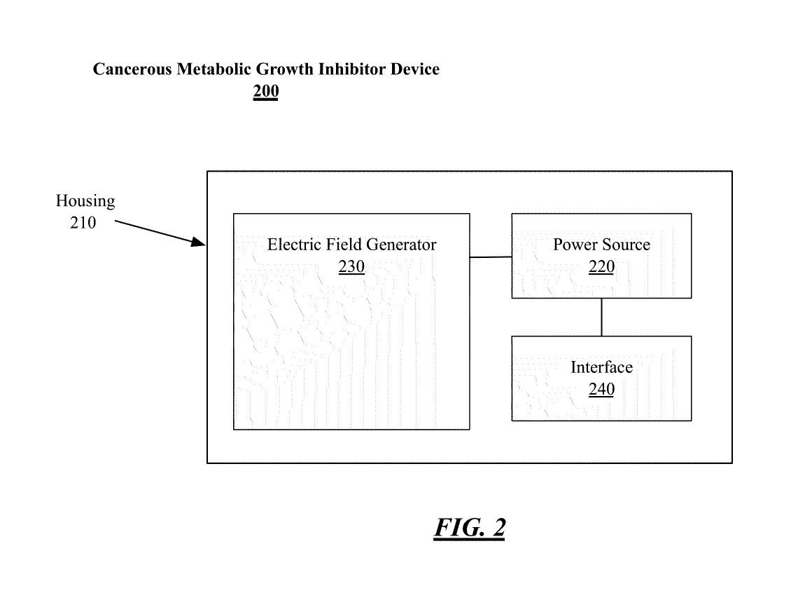

FIG. 2 illustrates an example medical device 200 to apply an electrical field to inhibit growth of target cells. As used herein, target cells are intended to mean the cells in which treatment is “targeted.” In some examples, the target cells will include one or more types of cancerous cells. Furthermore, as noted above, the present disclosure can be applied to treat tissue (sometimes referred to as “target tissue”) that includes both target and non-target cells. As used herein, non-target cells are intended to mean any cell that is not “targeted.” In some examples, non-target cells will include healthy cells, or more generally, non-cancerous cells. In some examples, the device 200 includes a housing 210, a power source 220, and an electric field generator 230. The power source 220 and the electric field generator 230 may be at least partially contained or “housed” within the housing 210. Furthermore, in some examples, the device 200 may include an interface 240.

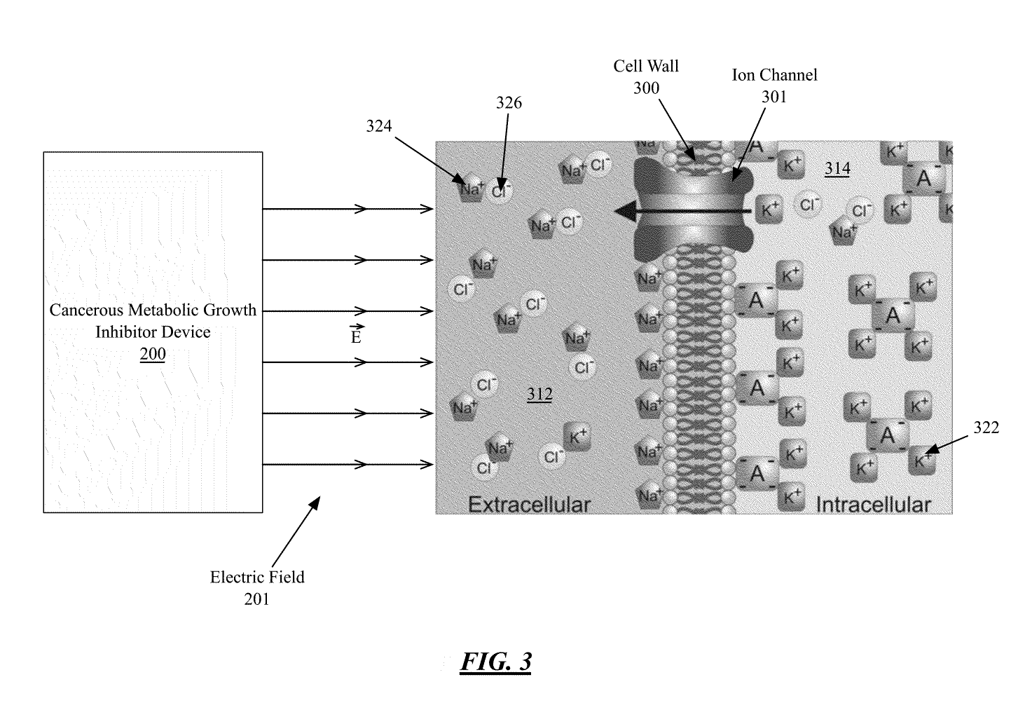

In general, the power source 220 is operably coupled to the electric field generator 230 to provide energy (e.g., electrical potential, or the like) to cause the electric field generator to generate an electric field that extends outside the housing 210 to encompass at least a portion of tissue to be treated. Said differently, the power source 220 may be electrically coupled to the electric field generator 230 to cause the electric field generator 230 to generate an electric field (e.g., refer to FIGS. 3-5) to be applied to target cells adjacent to the device 200.

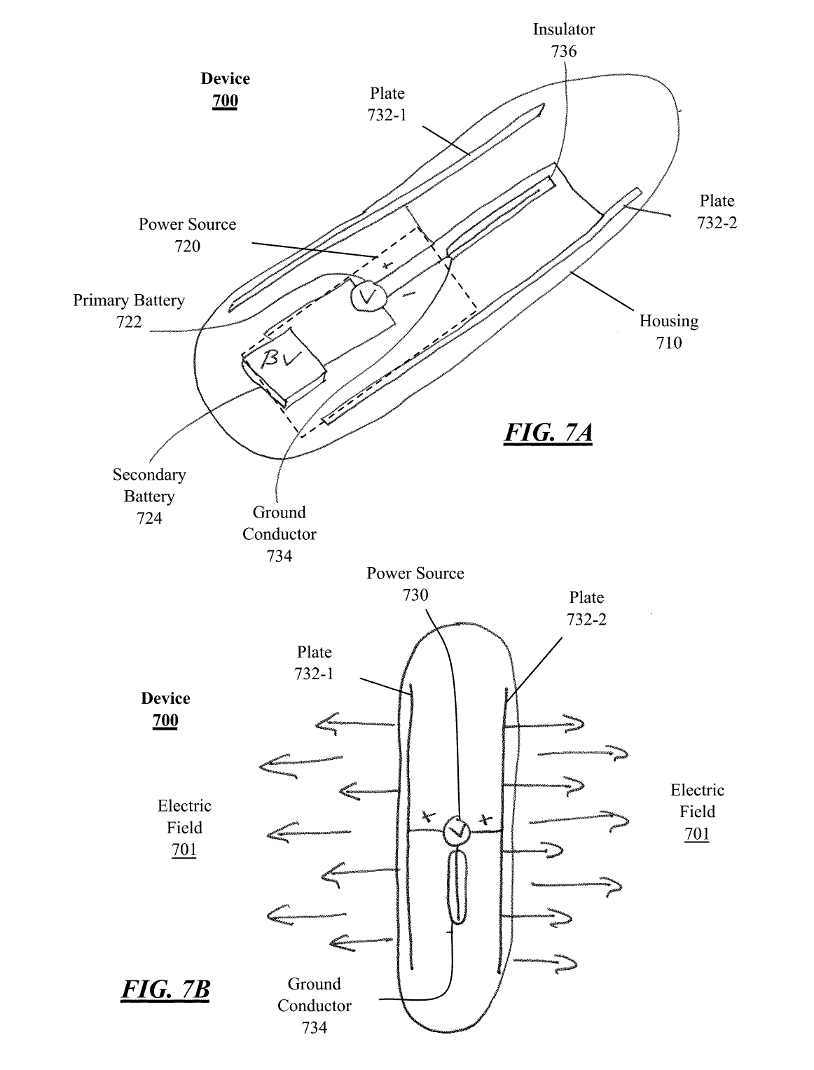

In general, the power source 220 may be any apparatus configured to output an electric potential. The power source 220 may be either a Direct Current (DC) power source or an Alternating Current (AC) power source. Furthermore, with some examples, the power source may include multiple power sources (e.g., refer to FIG. 7A). Additionally, the power source may be operably coupled to the interface 240, which may provide an external source of power to charge, increase, or otherwise add to the electrical potential available from the power source 220.

With some examples, the power source 220 may be characterized as a “low-voltage” power source. With some examples, the power source 220 may be configured to output an electrical potential sufficient to cause the electric field generator to apply between 2.0 V and 10.0 V to target tissue. With some examples, the power source 220 may be configured to output art electrical potential sufficient to cause the electric field generator 230 to apply between 2.0 V and 5.0 V to the target tissue. With some examples, the power source 220 may be configured to output an electrical potential sufficient to cause the electric field generator to apply between 2.0 V and 3.5 V to the target tissue. It is noted, that the present disclosure may be implemented to treat a variety of different types of cancer found in various locations throughout a body. In some examples, the electrical potential of the electric field may be based on the type of cancer being treated, and particularly, on the membrane potential of the target cells.

In general, the electric field generator 230 may be any apparatus configured to emit an electric field. With some examples, the electric field generator 230 may comprise a number of plates (e.g., refer to FIGS. 7A-7B, 9, and 11B), arranged to direct the electric field outside the housing 210. In some examples, the plates may be separated by an insulative element (e.g., refer to FIGS. 7A-7B, 9, and 11B), configured to insulate the plates from ground potentials between the plates. It is to be appreciated, that a number of different configurations of the electric field generator 230 can be implemented. As such, an attempt to exhaustively list them is not made here. In general, however, the electric field generator 230 is configured to emit an electric field outside the housing 210, such that the electric field can be applied to the target tissue. For example, the plates may be arranged in a parallel fashion, arranged in a circular fashion, arranged in a semi-circular fashion, arranged in a cone shaped fashion, or the like.

In some examples, the plates may be located within the housing 210. In some examples, the plates may be located outside the housing 210. In some examples, the plates may be located partially inside the housing 210 and partially outside the housing 210. Additional examples of the plates and their arrangement are given in FIGS. 7A-78, 9, and 11B.

In some examples, the electric field generator 230 may include coils, points, or other electrically conductive materials configured to emit an electric field when electric potential from the power source 220 is applied to the electrically conductive materials.

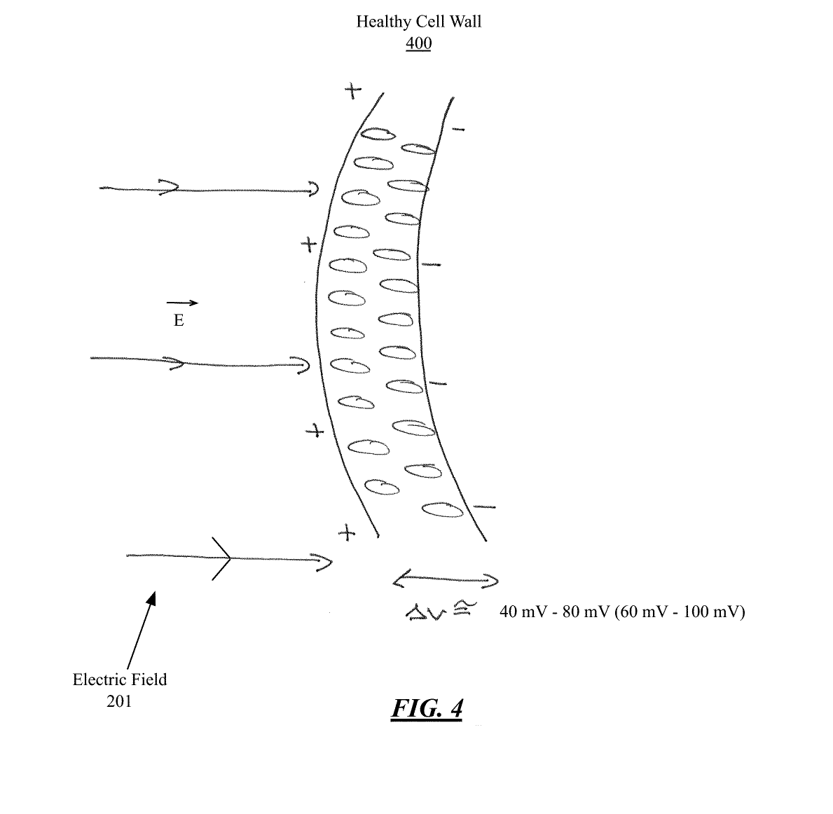

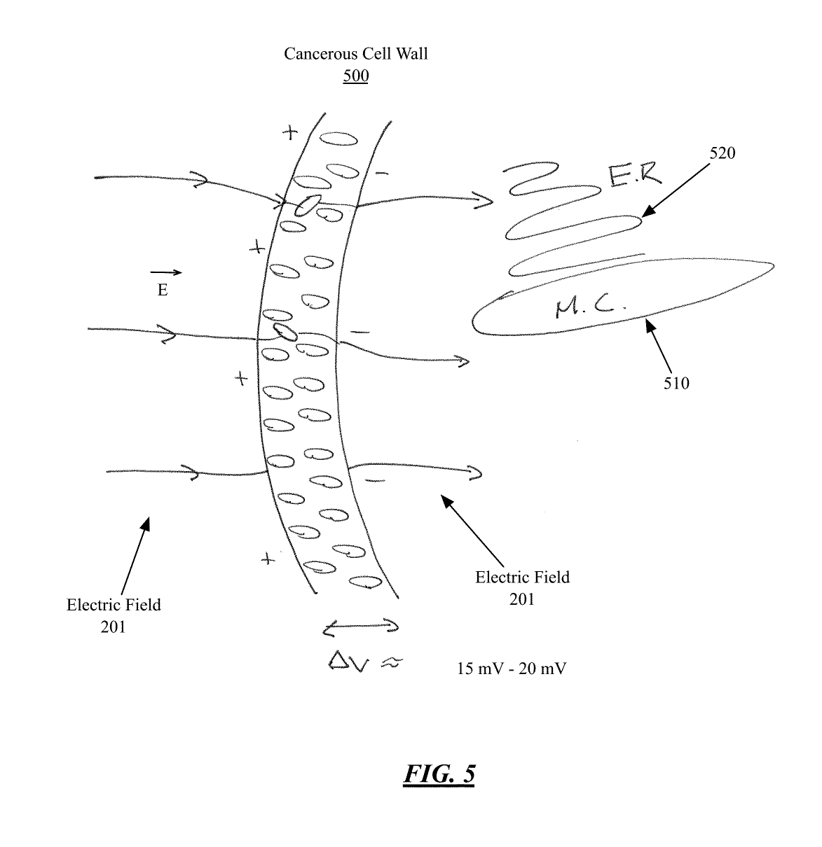

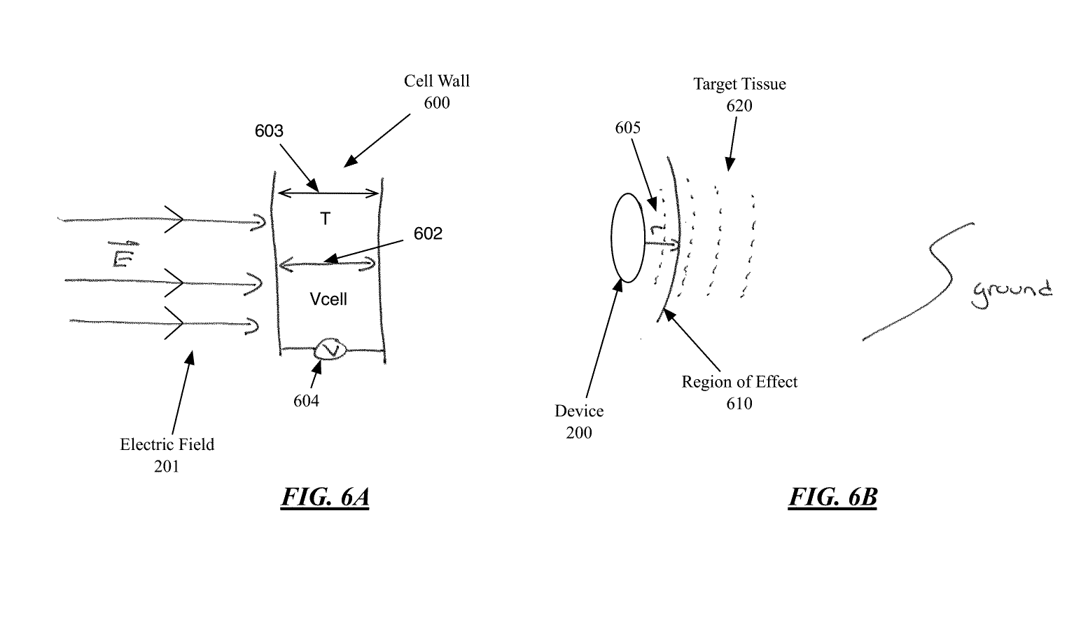

As described above, the device 200 can be implemented to apply an electric field to tissue that includes target cells (e.g., cancerous cells). In general FIGS. 3-5 and 6A-6B illustrate application of an electric field to cell walls to inhibit the growth of cancerous cells. More specifically, FIG. 3 illustrates an electric field applied to a cell wall and a membrane potential of the cell wall. FIG. 4 and FIG. 5 illustrate the effects of an electric field on a healthy cell and a cancerous cell, respectively. FIGS. 6A-6B illustrate the depth of penetration of an electric field through target tissue.

Turning more specifically to FIG. 3, a cell wall 300 under application of an electric field 201 from the device 200 is depicted. It is to be appreciated, that cell walls, such as the cell wall 300, have a membrane potential. In particular, the difference in the concentration of ions on opposite sides of the cell wall or “membrane” leads to an electric potential referred to as the “membrane potential.” It is noted, the cell wall 300 and the ion types and ion concentrations shown in this figure, are depicted for purposes of clarity of presentation and are not intended to be limiting. In practice, cell walls can have permeability to various types of ions, and as such, may have varying types and concentration of ions. Accordingly, the cells to which the present disclosure can be implemented to apply an electric field to inhibit the growth of cancerous cells is not intended to be limited by the cell wall depicted in this figure.

As depicted, the cell wall 300 includes an extracellular region 312 and an intracellular region 314. A number of ions have a concentration gradient across the cell wall 300. More particularly, a number of ions are concentrated in the extracellular region 312 and a number of ions are concentrated in the intracellular region 314. For example, potassium (K⁺) ions 322 are shown having a high concentration within the intracellular region 314 and a low concentration within the extracellular region 312; sodium (Na⁺) ions 324 and chloride (Cl⁻) ions 326 are shown at high concentrations within the extracellular region 312 and low concentrations in the intracellular region 314. These concentration gradients provide the potential energy to drive the formation of the membrane potential. The concentration can be affected by the ions moving across the cell wall (e.g., via transport processes or the like). An example of this is the sodium-potassium pump.

The voltage corresponding to the membrane potential is established when the cell wall 300 has permeability to one or more ions. For example, the cell wall 300 is depicted having an ion channel 301, which is permeable to potassium ions 322. Accordingly, potassium ions 372 can move from the intracellular region 314 to the extracellular region 312, thereby creating the membrane potential. It is noted, that the present disclosure references various ranges of membrane potential. These ranges are based on ranges specified in the references cite above. However, it is to be appreciated that these ranges are given for purposes of clarity of explanation and are not, necessarily limiting. In particular, the present disclosure may be provided to inhibit the growth of target cells having a different membrane potential than that used in the examples herein.

A non-cancerous or healthy cell has a membrane potential in the range of 40 mV to 80 mV (or 60 mV to 100 mV), while a typical cancer cell has a membrane potential in the range of 15 mV to 20 mV. The reduced membrane potential of cancer cells, with respect to normal cells, is due to the greatly increased cell division of a cancerous cell. More particularly, cancer cells depolarize, which is the mechanism whereby cancer cells undergo mitosis at a rate much higher than normal cells. The depolarization and increased division rate of cancer cells leads to the reduced membrane potential. Due to the reduced membrane potential of a cancer cell, an electric field (e.g., in the range of 1 to 40 mV) is capable of transporting through ion channels (e.g., the ion channel 301) within the target cells while not transporting through ion channels within non-target cells.

In particular, the electric field 201 may be configured such that an electric potential is applied to the cell wall 300 sufficient to overcome the reduced membrane potential of the target cells (e.g., cancerous cells) but not the non-target cells (e.g. non-cancerous cells). Accordingly, the electric field may transport through ion channels 301 of the target cells but not the non-target cells. As such, the electric field 201 may penetrate into the intracellular region 314 of target cells but not healthy cells, thereby inhibiting growth of the target cells but not the non-target cells. More specifically, penetration of the electric field into the intracellular region 314 of the target cells will disrupt the target cells ATP cycle, in essence “starving” the target cells of nutrients and causing a substantial reduction in cell mitosis. As detailed below, the affect of the electric field on the non-target cells may be small compared to the affect on the target cells. Furthermore, it is noted, that not all target cells may be affected. However, as the mitosis of the affected target cells is decreased, the overall growth of the target cells may be substantially reduced compared to non-treated target cells.

More specifically, FIG. 4 depicts the electric field 201 applied to treat a cell wall 400 corresponding to a non-target cell (e.g., a healthy cell). Due to the higher voltage range of the membrane potential of the non-target cell wall 400 (as compared to a target cells), the treatment electric field 201 cannot penetrate cell wall 400. Said differently, the potential energy of the treatment electric field 201 is insufficient to overcome the energy barrier (e.g., >40 mV, or the like) of the cell wall 400.

However, FIG. 5 depicts the electric field 201 applied to treat a cell wall 500 corresponding to a target cell (e.g., a cancerous cell). Due to the lower voltage range of the membrane potential of the cell wall 500 (e.g., as compared to non-target cells), with respect to a healthy cell, the treatment electric field 201 can penetrate the cell wall 500. Said differently, the potential energy of the treatment electric field 201 is sufficient to overcome the energy barrier (e.g., >15 mV, or the like) of the cell wall 500.

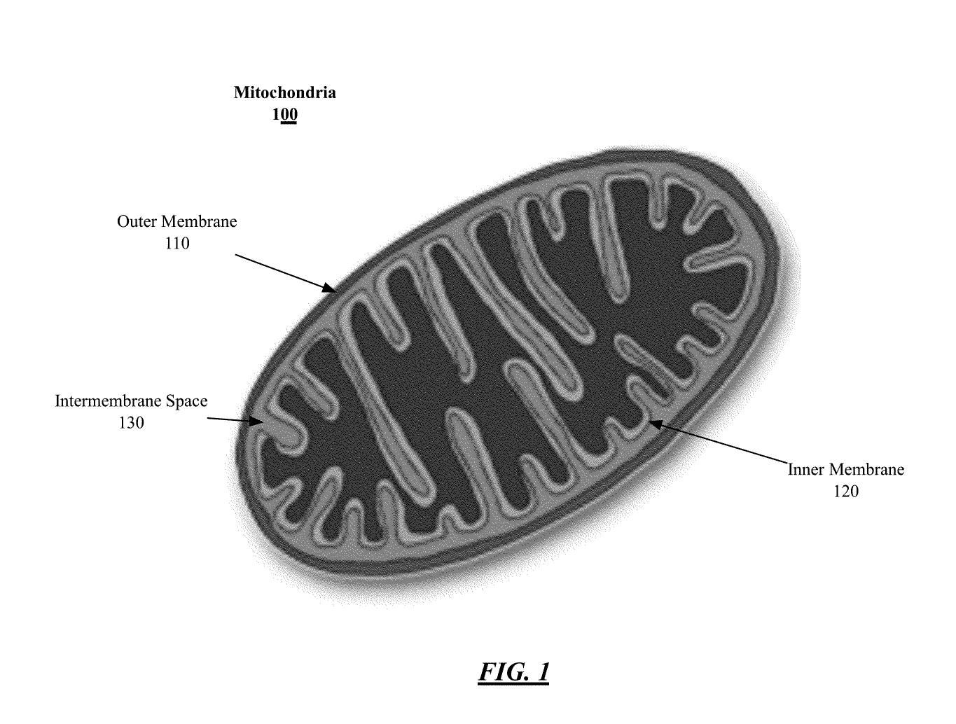

Once the electrical field 201 has penetrated the cell wall 500, the mitochondria (MC) 510 and the endoplasmic reticulum (ER) 520 as well as the mitochondria-associated membranes (MAM) (e.g., refer to FIG. 1) will be directly impacted by the electric field 201. More particularly, the electric field 201 may reduce the adenosine triphosphate (ATP) cycle capability of the cell. For example, the electric field 201 may interrupt functioning of various ion channels across the MAM (e.g., the Ca2+ channels, or the like). Such interruptions may lead to a reduced ATP production by the cell. It is noted, that during mitosis, ion channels may open, thereby enabling increased ATP activity. As such, application of an electric field to target tissue as described herein may cause a greater interruption to ATP production by cells that are undergoing mitosis.

Locally the application of an electric field to target tissue, and the range of effect of the electric filed can be modeled as the application of a voltage across a series of capacitors. Turning more specifically to FIG. 6A, the cell wall 600 is shown with the electric field 201 applied to the cell wall 600. The voltage drop 604 across the cell wall 600 is a function of the membrane potential (Cₑₗₗ) 602 of the cell wall 600. For example, if the cell wall 600 corresponds to a healthy cell, the voltage drop 604 would be a function of the membrane potential (e.g., 60 mV to 100 mV), whereas, if the cell wall 600 corresponds to a cancerous cell, the voltage drop 604 would be a function of the membrane potential (e.g., 15 mV to 20 mV).

Turning more specifically to FIG. 6B, the penetration depth of the electric field, and accordingly, the area of affected tissue, can be modeled as the work done by the electric field 201 as the electric field moves a charge a particular distance across a series of capacitors. In particular, the device 200 emitting an electric field into target tissue 620 will affect tissue within a region of effect 610. The radius (R) 605 of the region of effect 610 can be characterized by the strength of the electric field and the characteristics of the cells within the region 610 (e.g., cancerous or healthy. etc.). More specifically, the radius 605 of the region of effect 610 can be modeled using both the membrane potential 602 and the thickness (e.g., the thickness (T) 603 shown in FIG. 6A) of the cells within the region of effect 610.

In particular, the following equation for work done across a series of capacitors can be used to model the region of effect 610: −W=∫₀^{R}qE·dr=qV(r); where W is the work done by the battery (e.g., the power source 220, or the like), r is a distance from the origin of the electric field 201, qE is the force exerted by the electric field 201, and qV(r) is the work done by the electric field 201 to move the charge the distance R.

From the above equation, the distance R can be calculated using

$$R = \frac{{V}_{E}}{{V}_{cell}} T ;$$

where V_{E} is the applied voltage of the electric field 201 (or the energy potential of the electric field 201 ), V_{Cell} the membrane potential (e.g., 602 ) of the cell walls within the region 610 and T is the thickness (e.g., 603 ) of the cell walls within the region 610.

Assume that the electric field 201 emitted by the device 200 has an energy potential of 2 V, the target tissue 620 has membrane potentials of 100 mV (e.g., the target tissue is non-cancerous), and the average thickness of the cell walls in the tissue is 100 μm. Accordingly, the region of effect can be determined based on the above equations as follows:

$${D}_{Region of Effect} = 2 \frac{2 V}{100 mV} 100 μ m \sim 0.5 cm .$$

Accordingly, for non-cancerous cells, the device 200 emitting an electric field 201 having an energy potential of approximately 2 V, will affect tissue within a region of effect 610 having a diameter of approximately 0.5 cm.

As another example, assume that the target tissue 620 has membrane potentials of 15 mV (e.g., the target tissue is cancerous) and the average thickness of the cell walls in the tissue is 75 μm. It follows, the region of effect can be determined based on the above equations as follows:

$${D}_{Region of Effect} = 2 \frac{2 V}{15 mV} 75 μ m \sim 2.0 cm .$$

Accordingly, for cancerous cells, the device 200 emitting an electric field 201 having an energy potential of approximately 2 V, will affect tissue within a region of effect 610 having a diameter of approximately 2.0 cm.

Accordingly, the device 200, emitting the electric field 201, may interrupt the ATP cycles of healthy cells within a 0.5 cm diameter sphere and interrupt the ATP cycle of cancer cells within a 2.0 cm diameter sphere centered on the implant. It is to be appreciated, that this above example is given for purposes of illustration only and is not intended to be limiting. In particular, the membrane potential ranges, the cell wall thicknesses, and the strength of the electric field may be different for various implementations, and as such, are not intended to be limiting. Furthermore, it is to be appreciated, that the above examples are simplified and do not account for tissue containing both cancerous and healthy cells. Furthermore, it is to be appreciated, that as the region of effect 610 is linear in voltage, doubling the source voltage (e.g., the energy potential of the electric field, or the like) will double the size of the region of effect 610.

The duration of effect can also be modeled based on the tissue type (e.g., healthy, cancerous, or the like) in which the device 200 is placed. It is to be appreciated, that the insulated ground (e,g., refer to FIG. 7A-7B) within the device and/or power source may not serve as a power drain on the system. However the surface of the patient may serve as the nearest ground capable of draining the power source. For example, for human patients where the device is implanted, the resistance between the point of implantation and the point of ground could be between 1,000 ohms to 100,000 ohms. Accordingly, the functional current flow may be between 20 μA and 2 mA. The time to discharge of power source can be modeled using the following equation:

$$t = \frac{{Q}_{E}}{{I}^{k}} ,$$

where k is approximately 1.3 (battery chemistry constant), I is the current draw from the battery, and Q_{E} is the force exerted by the electric field referenced above. Additionally, it is to be appreciated, that during operation, the power source may lose some of its stored energy at a rate of 10-20 percent per year to self-discharge. Accordingly, the duration of effect may also be based on the amount the power source self discharges.

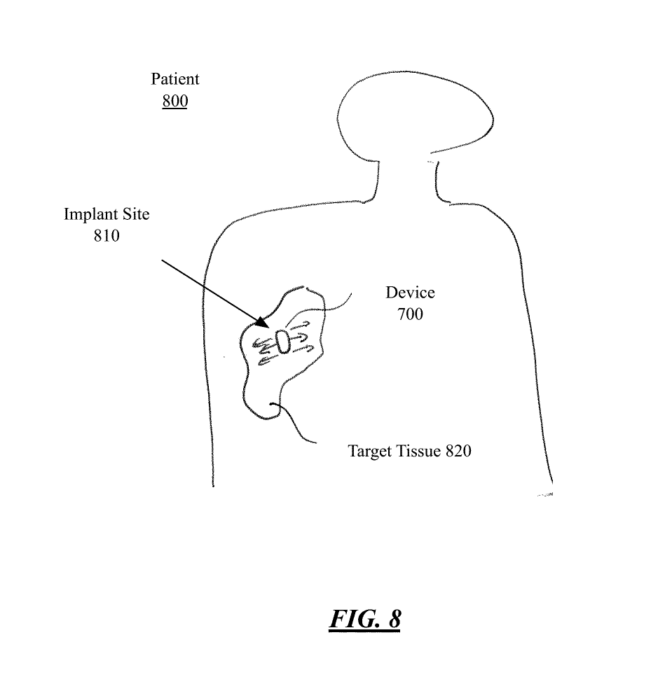

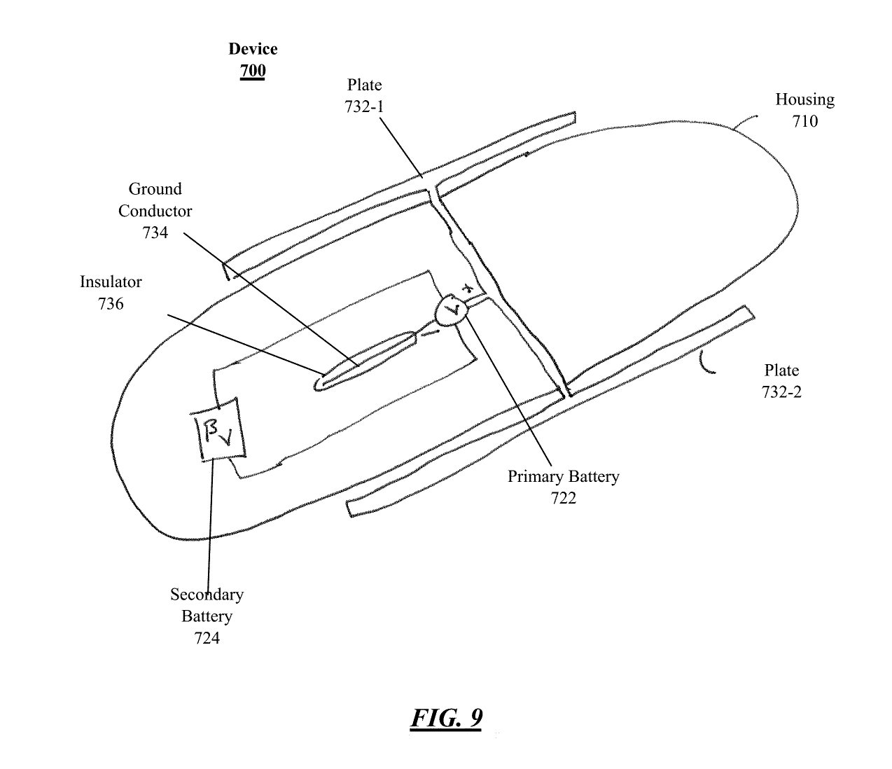

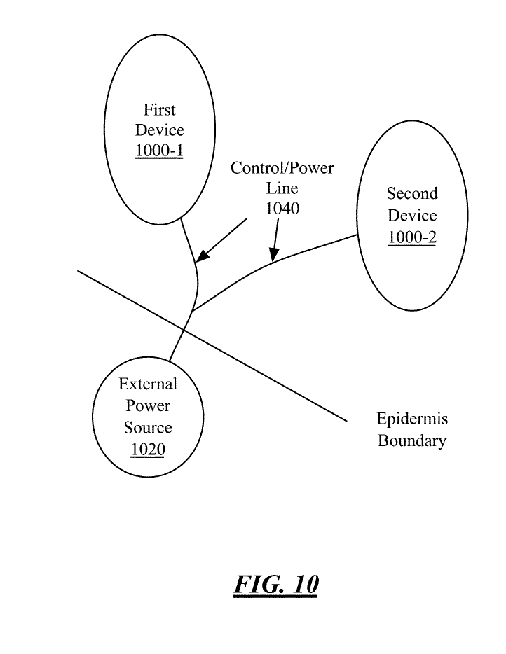

FIGS. 7A-7B and 8-9 illustrate an example implementation of an implantable medical device 700 to apply an electrical field to inhibit growth of cancerous cells. In some examples, the device 200 may be implemented as the device 700. In general, FIG. 7A-7B illustrates an example of the device 700, and FIG. 8 illustrates an examples of the device 700 implanted in a patent. FIG. 9 illustrates an alternative example of the implantable medical device 700. FIG. 10 illustrates an example of multiple devices 1000 implanted in a patient and operably coupled to an external power source.

Turning more specifically to FIG. 7A, the device 700 may include a housing 710, a power source 720, and an electric field generator (described in greater detail below). With some examples, the housing 710 may be “pill” shaped, as shown. In some examples, the housing 710 may be formed from a biocompatible coating (e.g., medical grade stainless steel, medical grade plastic, or the like) so as to not react with biological tissue. In some examples, the housing 710 may be thin, and may be non-conformable.

In some examples, the housing 710 may enclose all other components of the device 700. As such, the device 700 may be implantable to be placed in the center of a target site (refer to FIG. 8) by means of the delivery system (not shown). In some examples, one or more components of the device may be external to the housing 710 (e.g., refer to FIG. 9).

With some examples, the power source 720 may include a primary power source 722 and a secondary power source 724. In some examples, the primary power source 722 may be a battery having an energy potential of between 2 V and 3.5 V and a size of between 2 mm to 3 mm. The primary battery 722 may be operably coupled to the electric field generator to cause the electric field generator to emit an electric field (e.g., refer to FIG. 7B). The secondary battery 724 may be a battery configured to “trickle-charge” the primary battery 722. In particular, the secondary battery 724 may be operably coupled to the primary battery to cause the primary battery to not lose substantial energy potential due to “self-discharge.” For example, with some embodiments, the secondary battery 724 may be a betavoltaic battery. In other examples, the power source may include a single battery, which may be rechargeable (e.g., via radio frequency charging, or the like).

The power source 720 is configured to supply a voltage to the electric field generator. The electric field generator is represented by components 732-a, where “a” is a positive integer. For example, the device 700 is depicted including plates 732-1 and 732-2 that form part of the electric field generator. Furthermore, the electric field generator may include a ground conductor 734. It is to be appreciated, that the plates 732-1 and 732-2 shown here are shown for purposes of illustration only and not intended to be limiting. In practice, more or less plates 732-a than shown may be implemented. Furthermore, the arrangement of the plates 732-a and the placement of the ground conductor 734 may be such to cause the device 700 to emit an electric field outward from the housing 710. In some examples, the electric field generator may also include an insulator 736, to insulate the plates 732-1 and 732-2 from the ground conductor 734. Such insulation may be beneficial to cause the electric field to be emitted outside the housing, prevent conduction of current between plates, and/or reduce the amount of self-discharge of the power source 270.

Turning more specifically to FIG. 7B, the device 700 is illustrated emitting the electric field 701. In particular, this figure shows a cut-away view of the device 700. The power source 720, the plates 732-1 and 732-2, the ground conductor 734 are shown. The power source provides a voltage to cause the electric field generator to emit the electric field 701. It is to be appreciated, as described above, the plates 732-1 and 732-2 and the ground conductor 734 can be arranged in any fashion to cause the device 700 to emit the electric field 701 outward from the device as shown. As such, the electric field may be applied to tissue adjacent to the implant site. In some examples, the plates 732-1 and 732-2 may be configured to emit the electric field 701 in a particular direction. As such, tissue on a particular side or portion of the device 700 may be targeted.

Turning more specifically to FIG. 8, an example patient 800, with the device 700 implanted into a portion of the patient's body is shown. In particular, the device 700 is shown implanted at an implant site 810. The implant site 810 is within a portion of target tissue 820. The target tissue 820 may correspond to cancerous tissue (e.g., tumorous tissue, or the like) or may correspond to a region of tissue there the prevention of tumor seeding is desired. The device 700 may be implanted using a deliver system to avoid open surgery or otherwise invasive procedures. For example, the device 700 may be implanted using a trocar, hollow needle, or a delivery handle with a push bar, or the like. Once in location, the device 700 can be deployed into patient's body to the target location 810.

The device 700 may be placed at the target site 810 using one or more imaging modalities to ensure the device 700 is placed at the proper location. Furthermore, in some examples, the delivery system used to implant the device 700 may be configured to emit an electric field during implantation, and more specifically, post implantation (e.g., as the delivery system component is withdrawn from the patient's body), to moderately ablate the entry line to prevent tumor seeding along the entry points. For example, the delivery system may include an electric field generator (e.g., as described herein) to moderately ablate the entry line during withdrawal of the delivery system components from the patient's body.

Turning more specifically, to FIG. 9, the device 700 is shown. However, as depicted, portions of the device 700 are external to the housing 710. In particular, the plates 732-1 and 732-2 protrude from the housing 710. In such an example, plates 732-a may be formed from and/or coated with a biocompatible material. For example, the device 700 may be configured such that the plates 732-1 and 732-2 protrude from the housing 710 such that the housing 710 may be smaller relative to a similar device where the plates are internal to the housing. Additionally, the plates 732-1 and 732-2 may be configured to cooperate with plunger and/or pusher devices to deploy the device into a patient's body.

Turning more specifically to FIG. 10, multiple devices 1000-a are shown implanted into a patient, where each device is operably coupled to an external power source. In particular, devices 1000-1 and 1000-2 are shown. The devices 1000-a may be substantially similar to the device 700 described above, except that the power source is shown externally connected to the devices 1000-a. In particular, the devices 1000-1 and 1000-2 may be implanted at a first target site and a second target site, respectively, control and/or power lines 1040 may be routed to an externally connected power source 1020. In particular, the devices 1000-1 and 1000-2 may be implanted into a patient's body, while a power source is kept outside the patient's body (e.g., outside the epidermis boundary, or the like).

In some examples, the external power source 1020 may be worn and/or carried by the patient. Although such an embodiment may result in increased patient complications due to lines 1040 extending outside the body surface, (e.g., due to the lines 1040) the external power source may be configured to output a voltage greater than possible using internal power sources. Furthermore, multiple devices (e.g., as shown) may be powered. Additionally, the electric fields generated by each device may be combined and/or controlled by adjusting the voltage and/or polarity of the external power source 1020.

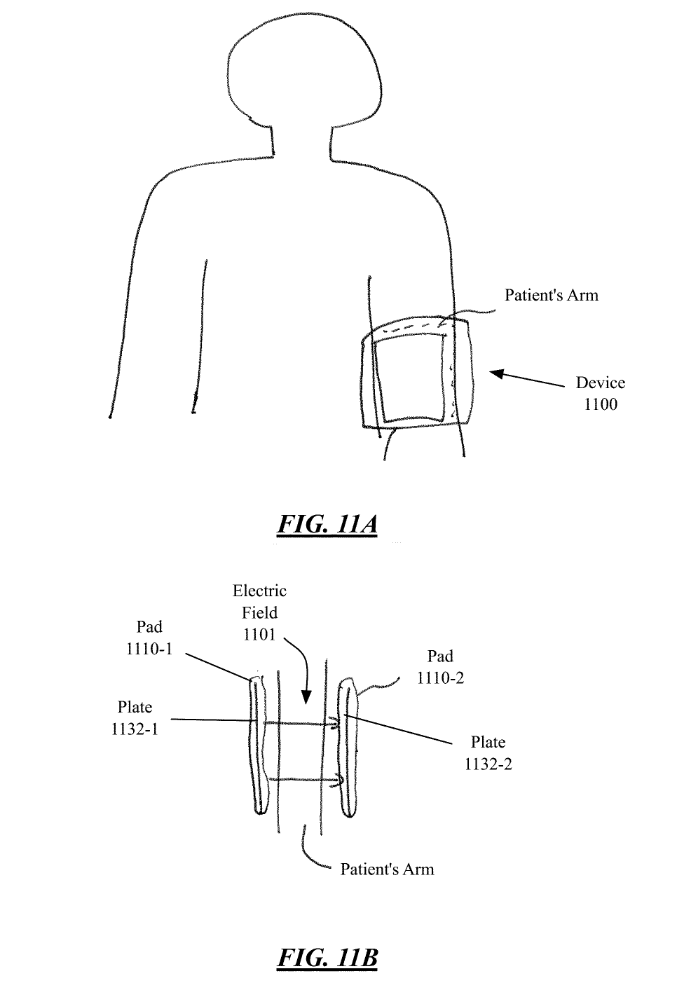

FIGS. 11A-11D illustrate an example implementation of a wearable medical device 1100 to apply an electrical field to inhibit growth of cancerous cells. In some examples, the device 200 may be implemented as the device 1100. In general, FIG. 11A illustrates the device worn on a patient's arm while FIG. 11B illustrates a cut-away view of the device 1100 worn on the patient's arm showing the electric field applied to tissue in the patients arm.

In particular, the device 1100 is shown including pads 1110-a that may each include a plate or plates to form an electric field. In particular, pads 1110-1 and 1110-2 are shown including plates 1132-1 and 1132-2, respectively. The pads 1110-1 and 1110-2 may be made of a flexible material suitable to be worn by a patient. For example, as shown, the pads 1110-1 and 1110-2 are worn on a patient's limb, specifically, an arm. It is to be appreciated that the pads may be designed to be worn, attached, or placed near any portion of a patient's body. Furthermore, any number and scale of pads could be included in the device 1100 to produce an electric field having sufficient enemy potential to affect a desired tissue mass. The device 1100 may be operably coupled to a power source (e.g., similar to the external power source 1040 described above) to cause the plates 1132-1 and 1132-2 to produce the electric field 1101. In some examples, the pads 1110-1 and 1110-2 may include an insulative layer to prevent current flow between the plates 1132-1 and 1132-2.

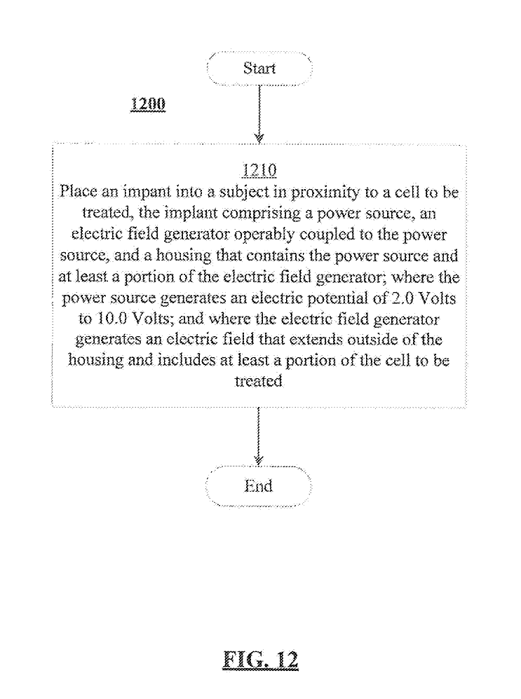

FIG. 12 illustrates a method 1200 for treating a cell located within a living subject. The method 1200 may begin at block 1210. As block 1210, an implant may be placed into a subject in proximity to a cell to be treated; the implant comprising a power source, an electric field generator operably coupled to the power source, and a housing that contains the power source and at least a portion of the electric field generator; where the power source generates an electric potential of between 2.0 to 10.0 Volts; and where the electric field generator generates an electric field that extends outside of the housing and includes at least a portion of the cell to be treated. For example, at block 1210, the device 700 may be implanted into a patient's body as shown in FIG. 8.

1. A method of treating a cancer cell, comprising the steps of:
placing a device into the subject and in proximity to the cancer cell, said device comprising a power source, an electric field generator operably coupled to the power source, and a housing that contains the power source and at least a portion of the electric field generator;
wherein the power source generates an electric potential of up to 10.0 Volts; and
wherein the electric field generator generates an electric field that extends outside of the housing and includes at least a portion of the cancer cell.

2. The method of claim 1, comprising activating the power source.

3. The method of claim 1, wherein the power source comprises a betavoltaic battery.

4. The method of claim 1, wherein the power source is a direct current power source.

5. The method of claim 1, wherein the power source is an alternating current power source.

6. The method of claim 1, wherein the electric field generator comprises two plates arranged to direct the electric field outside the device.

7. The method of claim 6, wherein the device comprises a ground conductor arranged to direct the electric field outside the device.

8. The method of claim 7, wherein the device comprises an insulative element to insulate the two plates from the ground conductor.

9. The method of claim 6, wherein the plates are located inside of the housing.

10. The method of claim 6, wherein the plates are located outside of the housing.

11. The method of claim 1, wherein the electric field generator comprises a coil.

12. The method of claim 1, wherein the electric field generator generates an electric field characterized by a voltage of 2.0 Volts to 5.0 Volts.

13. The method of claim 1, wherein the device further comprises at least one wire in electrical connection to the power source and extending outside of the housing, the wire configured to be electrically connected to an external power source located outside of the living subject.

14. The method of claim 1, wherein the device further comprises a receiving coil located outside of the living subject, the receiving coil in electrical communication with the power source.

15. The method of claim 1, wherein said housing comprises a material that is substantially non-conductive of electric current.

16. The method of claim 1, wherein said housing comprises a polymeric material.

17. A method of treating a cancer cell, comprising the steps of:
placing a device into the subject and in proximity to the cancer cell, said device comprising a power source, an electric field generator in electrical connection to the power source, and a housing that contains the power source and at least a portion of the electric field generator;
wherein the power source genrates an electric potential of 2.0 Volts to 3.5 Volts; and
wherein the electric field generator comprises a plurality of plates arranged to generate an electric field that extends outside of the housing and includes at least a portion of the cancer cell.

18. The method of claim 17, comprising treating the cancer cell using one or more secondary treatment modalities.

19. A method of treating a cancer cell, comprising the steps of:
placing a device into the subject and in proximity to the tissue to be treated, said device comprising a power source, an electric field generator in electrical connection to the power source, and a housing that contains the power source and at least a portion of the electric field generator:
wherein the power source comprises a primary battery and a secondary battery, the primary battery to generate an electric potential of 2.0 Volts to 5.0 Volts; and
wherein the electric field generator generates an electric field that extends outside of the housing and includes at least a portion of the cancer cell.

20. The method of claim 19, wherein the secondary battery comprises a betavoltair battery.
